SEQUENCE LISTING (1 sequence)

SEQ ID NO: 1 - protein, 8 aa, Artificial Sequence
  Synthetic
GPHHHHHH

(19) United States
(12) Patent Application Publication
(10) Pub. No.: US 2023/0285405 A1
(43) Pub. Date: Sep. 14, 2023

(54) Pharmaceuticals for Treating or Preventing Nidoviruses and Picornaviruses

(71) Applicant: The University of Chicago, Chicago, IL (US)

(72) Inventors: Savas Tay, Chicago, IL (US); Glenn Randall, Chicago, IL (US); Nir Drayman, Evanston, IL (US); Siquan Chen, Burlingame, CA (US)

(73) Assignee: The University of Chicago, Chicago, IL (US)

(21) Appl. No.: 18/019,972

(22) PCT Filed: Aug. 6, 2021
(86) PCT No.: PCT/US2021/044971

§ 371 Date: Feb. 6, 2023

Related U.S. Application Data
(60) Provisional application No. 63/062,775, filed on Aug. 7, 2020.
(60) Provisional application No. 63/127,436, filed on Dec. 18, 2020.

(51) Int. Cl. A61K 31/55; A61P 31/14

(52) U.S. Cl. CPC A61K 31/55; A61P 31/14

(57) ABSTRACT

In aspects, the invention provides a method of treating or preventing a nidovirus or picornavirus infection in a subject, the method comprising administering to a subject in need thereof an effective amount of an active agent of butamben, butylparaben, conivaptan, amphotericin B, pentoxyverine, lapatinib, vilazodone, imatinib (STI571), benztropine, raloxifene, solifenacin, retapamulin, bafetinib (INNO-406), imipramine, trimipramine, tolterodine, clomipramine, velpatasvir, cediranib (AZD2171), azelastine, desloratadine, nortriptyline, propafenone, mebeverine, elbasvir, cepharanthine, flupenthixol dihydrochloride, or a pharmaceutically acceptable salt thereof.

### CROSS REFERENCE TO RELATED APPLICATION

This patent application claims the benefit of U.S. Provisional Patent Application No. 63/062,775, filed Aug. 7, 2020, and U.S. Provisional Patent Application No. 63/127,436, filed Dec. 18, 2020, each of which is herein incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 493 Byte ASCII (Text) file named “756710_ST25” dated Aug. 3, 2021.

### BACKGROUND OF THE INVENTION

The current global COVID-19 pandemic is caused by the coronavirus SARS-CoV-2, which is a nidovirus. There is a current need for treatment and prevention of nidoviruses, including coronaviruses, as well as picornaviruses, which are in the same class of viruses as nidoviruses.

### BRIEF SUMMARY OF THE INVENTION

In aspects, the invention provides a method of treating or preventing a nidovirus or picornavirus infection in a subject, the method comprising administering to a subject in need thereof an effective amount of an active agent of butamben, butylparaben, conivaptan, amphotericin B, pentoxyverine, lapatinib, vilazodone, imatinib (STI571), benztropine, raloxifene, solifenacin, retapamulin, bafetinib (INNO-406), imipramine, trimipramine, tolterodine, clomipramine, velpatasvir, cediranib (AZD2171), azelastine, desloratadine, nortriptyline, propafenone, mebeverine, elbasvir, cepharanthine, flupenthixol dihydrochloride, or a pharmaceutically acceptable salt thereof.

Additional aspects are as described herein.

### BRIEF DESCRIPTION OF THE DRAWINGS

FIG. 1A presents dose response curves of remdesivir and top hits (amphotericin B, elbasvir, cediranib, and cepharanthine) from the screen described in Example 1, n=3. Individual measurements are shown as semi-transparent circles (some circles overlap).

FIG. 1B presents dose response curves of additional top hits (butylparaben, flupenthixol, velpatasvir, and bafetinib) from the screen described in Example 1, n=3. Individual measurements are shown as semi-transparent circles (some circles overlap).

FIG. 1C presents dose response curves of additional top hits (raloxifene, clomipramine, butamben, lapatinib, and desloratadine) from the screen described in Example 1, n=3. Individual measurements are shown as semi-transparent circles (some circles overlap).

FIGS. 2A and 2B present graphs showing results of treatment of A549 cells over-expressing ACE2 pre-treated with the indicated drugs (remdesivir, cepharanthine, flupenthixol, desloratidine, and trimipramine in FIG. 2A; lapatinib, benztropine, bafetinib, and azelastine in FIG. 2B) for 2 hours, infected with SARS-CoV-2 (MOI 0.5) and incubated for 2 days. Cells were stained for the presence of the spike protein and the percent of infected cells was analyzed. Most of the drugs effective against OC43 showed similar effectivity against SARS-CoV-2, n=3. Individual measurements are shown as semi-transparent circles (some circles overlap).

FIG. 2C is a graph showing the effect of selected drugs on SARS-CoV-2 progeny production. Cells were treated with 10 μM of the indicated drugs for 2 hours, infected with SARS-CoV-2 (MOI 0.5) and cell supernatant were collected for titration 2 days later, n=3. Individual measurements are shown as semi-transparent circles. All drugs showed a statistically significant (p-values<0.001, one-tailed t-test, FDR-corrected) reduction in viral titers.

FIG. 3 is a bar graph showing results of a FlipGFP reporter assay performed to screen for potential inhibition of 3CLpro by the identified drugs at a single concentration (10 μM). Shown are the drugs that showed a statistically significant reduction in 3CLpro activity (p-value <0.05, one-tailed t-test, FDR-corrected). n=6. The data for the remaining tested drugs is shown in FIG. 7. Individual measurements are shown in circles. Bars depict mean±s.e.

FIGS. 4A-4G present line graphs showing the effect of drugs on cell growth (% of no drug controls). Mean±S.E. n=3. FIG. 4A shows the results for butamben, butylparaben, flupenthixol, and imipramine. FIG. 4B shows the results for nortriptyline, propafenone, solifenacin, and conivaptan. FIG. 4C shows the results for vilazodone, azelastine, desloratadine, and retapamulin. FIG. 4D shows the results for raloxifene, clomipramine, velpatasvir, and amphotericin B. FIG. 4E shows the results for benztropine, tolterodine, bafetinib, and imatinib. FIG. 4F shows the results for mebeverine, trimipramine, pentoxyverine, and cepharanthine. FIG. 4G shows the results for elbasvir, erythromycin, remdesivir, cediranib, and lapatinib.

FIGS. 5A-5C present dose response curves of certain tested drugs, n=3. Individual measurements are shown as semi-transparent circles (some circles overlap). FIG. 5A shows the results for vilazodone, azelastine, trimipramine, retapamulin, and conivaptan. FIG. 5B shows the results for solifenacin, pentoxyverine, propafenone, nortriptyline, and imatinib. FIG. 5C shows the results for benztropine, mebeverine, imipramine, tolterodine, and erythromycin.

FIGS. 6A-6D present dose response curves of the certain tested drugs, n=3. Individual measurements are shown as semi-transparent circles (some circles overlap). FIG. 6A shows the results for clomipramine, notryptyline, solifenacin, pentoxyverine, and cedrianib. FIG. 6B shows the results for propafenone, raloxifene, vilazodone, imatinib, and conivaptan. FIG. 6C shows the results for retapamulin, mebeverine, elbasvir, velpatasavir, and butamben). FIG. 6D shows the results for butylparaben and amphotericin B.

FIG. 7 is a bar graph showing the results of a FlipGFP reporter assay performed to screen for potential inhibition of 3CLpro by the identified drugs at a single concentration (10 μM). Shown are all the drugs that did not show a significant reduction in 3CLpro activity (p-value >0.05, one-tailed t-test, FDR-corrected). n=6. Individual measurements are shown in circles. Bars depict mean±s.e. The drugs that showed statistically significant inhibition of 3CLpro are shown in FIG. 3.

### DETAILED DESCRIPTION OF THE INVENTION

In aspects, the invention provides a method of treating or preventing a nidovirus or picornavirus infection in a subject, the method comprising administering to a subject in need thereof an effective amount of an active agent of butamben, butylparaben, conivaptan, amphotericin B, pentoxyverine, lapatinib, vilazodone, imatinib (STI571), benztropine, raloxifene, solifenacin, retapamulin, bafetinib (INNO-406), imipramine, trimipramine, tolterodine, clomipramine, velpatasvir, cediranib (AZD2171), azelastine, desloratadine, nortriptyline, propafenone, mebeverine, elbasvir, cepharanthine, flupenthixol, or a pharmaceutically acceptable salt thereof.

In aspects, conivaptan is conivaptan HCl, pentoxyverine is pentoxyverine citrate, vilazodone is vilazodone HCl, benztropine is benztropine mesylate, raloxifene is raloxifene HCl, solifenacin is solifenacin succinate, imipramine is imipramine HCl, trimipramine is trimipramine maleate, tolterodine is tolterodine tartrate, clomipramine is clomipramine HCl, azelastine is azelastine HCl, nortriptyline is nortriptyline HCl, mebeverine is mebeverine HCl, and flupenthixol is flupenthixol dihydrochloride.

In any of the aspects of the methods described herein, the nidovirus can be a coronavirus, an arterivirus, or a torovirus. In aspects, the nidovirus is a coronavirus, such as an alpha- or a beta-coronavirus. In aspects, the beta-coronavirus is a βA, βB, βC, or βD coronavirus. In aspects, the beta-coronavirus is HCoV-OC43, SARS-CoV, SARS-CoV-2, or MERS-CoV. Preferably, the beta-coronavirus is SARS-CoV-2. In aspects, the nidovirus is an arterivirus. In aspects, the nidovirus is a torovirus (e.g., renitovirus). In any of the aspects of the methods described herein, the picornavirus can be, e.g., polioviruses, rhinoviruses, enteroviruses and coxsackieviruses.

The disease caused by the nidovirus can be, for example, coronavirus disease (COVID-19), severe acute respiratory syndrome (SARS) virus, Middle East respiratory syndrome (MERS), a respiratory disease (e.g., pneumonia, bronchitis, pleural effusion), an inflammatory disease (e.g., inflammation, COVID-19-induced inflammation, pediatric multi-system inflammatory syndrome (PMIS)), reproductive and respiratory syndrome virus (PRRSV), equine arteritis virus (EAV), or gastroenteritis. The disease caused by the picornavirus can be, for example, acute flaccid myelitis (AFM), respiratory disease, and gastrointestinal disease.

The methods described herein can comprise using (e.g., administering) the active agent in the form of a pharmaceutical composition. In particular, a pharmaceutical composition will comprise at least one active agent, as described herein, and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the active compounds and one that has no detrimental side effects or toxicity under the conditions of use.

The pharmaceutical compositions may be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the pharmaceutical composition is administered orally or intravenously.

In accordance with any of the aspects, the active agent may be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms may be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The active agent may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as polyethylene glycol (e.g., PEG400), an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which may be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (e) mixtures thereof.

The parenteral formulations will typically contain from about 0.5 to about 25% by weight of the active agent in solution. Suitable preservatives and buffers may be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and may be stored in a freeze-dried (lyophilized) condition requiring only the addition of the sterile liquid carrier, for example, water, for injections, immediately prior to use. Extemporaneous injection solutions and suspensions may be prepared from sterile powders, granules, and tablets of the kind previously described.

The active agent may be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In aspects, the composition contains at least one active component and a suitable vehicle or carrier. It may also contain other components, such as an anti-irritant. The carrier may be a liquid, solid or semi-solid. In aspects, the composition is an aqueous solution, such as a mouthwash. Alternatively, the composition may be a dispersion, emulsion, gel, lotion or cream vehicle for the various components. In aspects, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions may be produced as solids, such as powders or granules. The solids may be applied directly or dissolved in water or a biocompatible solvent prior to use to form a solution that is substantially neutral or that has been rendered substantially neutral and that can then be applied to the target site. In aspects of the invention, the vehicle for topical application to the skin can include water, buffered solutions, various alcohols, glycols such as glycerin, lipid materials such as fatty acids, mineral oils, phosphoglycerides, collagen, gelatin, and silicone-based materials.

The active agent, alone or in combination with other suitable components, may be made into aerosol formulations to be administered via inhalation. These aerosol formulations may be placed into pressurized acceptable propellants. Suitable propellants include, e.g., a fluorinated hydrocarbon (e.g., trichloromonofluoromethane, dichlorodifluoromethane, chlorodifluoromethane, chlorodifluoroethane, dichlorotetrafluoroethane, heptafluoropropane, tetrafluoroethane, difluoroethane), a hydrocarbon (e.g., propane, butane, isobutane), or a compressed gas (e.g., nitrogen, nitrous oxide, carbon dioxide). They also may be formulated as pharmaceuticals for non-pressured preparations, such as in a nebulizer or an atomizer.

The dose administered to the subject, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular active agent and the desired effect. It will be appreciated by one of skill in the art that various conditions or disease states may require prolonged treatment involving multiple administrations.

The inventive methods comprise using an effective amount of the active agent. An “effective amount” means an amount sufficient to show a meaningful benefit in an individual, cell, or tissue to be treated. A meaningful benefit includes, for example, detectably treating, relieving, or lessening one or more symptoms of a disease caused by a nidovirus or picornavirus (e.g., inflammation, fluid accumulation), inhibiting, arresting development, preventing, or halting further development of the viral infection or disease, reducing the incidence of a disease caused by nidovirus or picornavirus, preventing a disease caused by nidovirus or picornavirus from occurring in a subject, cell, or tissue at risk thereof but yet to be diagnosed, and/or detectably inhibit one or more active sites of viral proteins in a subject, cell, or tissue. The meaningful benefit observed in the subject, cell, or tissue to be treated may be to any suitable degree (10, 20, 30, 40, 50, 60, 70, 80, 90% or more). In some aspects, one or more symptoms of the disease are prevented, reduced, halted, or eliminated subsequent to administration of an active agent described herein, thereby effectively treating the disease to at least some degree.

Effective amounts may vary depending upon the biological effect desired in the individual, cell and/or tissue to be treated, condition to be treated, and/or the specific characteristics of the active agent. In this respect, any suitable dose of the active agent may be administered to the subject (e.g., human), cell, or tissue. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the active agent desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints may be used to define a close-ended range, or a single endpoint may be used to define an open-ended range.

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of viral infection in a mammal. Furthermore, the treatment or prevention provided by the inventive method can include treatment or prevention of one or more conditions or symptoms of the infection being treated or prevented. For example, treatment or prevention can include promoting the reduction of viral titers. Also, for purposes herein, “prevention” can encompass delaying the onset of the infection, or a symptom or condition thereof. Alternatively or additionally, “prevention” may encompass preventing or delaying the recurrence of infection, or a symptom or condition thereof.

For purposes of the present invention, the term “subject” preferably is directed to a mammal. Mammals include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simioids (monkeys) or of the order Anthropoids (humans and apes). An especially preferred mammal is the human.

A subject in need thereof is any one that has come in contact with, suspected to have come in contact with, or expected to come into contact with a nidovirus (e.g., SARS-CoV-2) or picornavirus. At risk subjects for developing a disease caused by a nidovirus include, for example, people aged 40 and older (particularly people aged 60 and older), people with one more underlying conditions (e.g., cardiovascular disease, diabetes, chronic respiratory disease, asthma, liver disease, chronic kidney disease undergoing dialysis, high blood pressure, obesity (e.g., a body mass index (BMI) of 30 or higher, especially 40 or higher), and cancer), people that are immunocompromised (e.g., due to a condition such as smoking, cancer treatment, bone marrow or organ transplantation, HIV, AIDS, and prolonged use of corticosteroids and other immune weakening treatments), and people living in a nursing home or a long-term care facility.

The following includes certain aspects of the invention.
1. A method of treating or preventing a nidovirus or picornavirus infection in a subject, the method comprising administering to a subject in need thereof an effective amount of an active agent of butamben, butylparaben, conivaptan, amphotericin B, pentoxyverine, lapatinib, vilazodone, imatinib (STI571), benztropine, raloxifene, solifenacin, retapamulin, bafetinib (INNO-406), imipramine, trimipramine, tolterodine, clomipramine, velpatasvir, cediranib (AZD2171), azelastine, desloratadine, nortriptyline, propafenone, mebeverine, elbasvir, cepharanthine, flupenthixol, or a pharmaceutically acceptable salt thereof.
2. The method of aspect 1, wherein conivaptan is conivaptan HCl, pentoxyverine is pentoxyverine citrate, vilazodone is vilazodone HCl, benztropine is benztropine mesylate, raloxifene is raloxifene HCl, solifenacin is solifenacin succinate, imipramine is imipramine HCl, trimipramine is trimipramine maleate, tolterodine is tolterodine tartrate, clomipramine is clomipramine HCl, azelastine is azelastine HCl, nortriptyline is nortriptyline HCl, mebeverine is mebeverine HCl, and flupenthixol is flupenthixol dihydrochloride.
3. The method of aspect 1, wherein the active agent is azelastine.
4. The method of any one of aspects 1-3, wherein the virus is a nidovirus and the nidovirus is a coronavirus.
5. The method of aspect 4, wherein the coronavirus is a beta-coronavirus.
6. The method of aspect 5, wherein the beta-coronavirus is SARS-CoV-2.
7. The method of any one of aspects 1-6, wherein the method is a method of treating.
8. The method of any one of aspects 1-7, wherein the subject is a human.

It shall be noted that the preceding are merely examples of aspects of the present disclosure. Other exemplary aspects are apparent from the entirety of the description herein. It will also be understood by one of ordinary skill in the art that each of these aspects may be used in various combinations with the other aspects provided herein.

The following examples further illustrate the present disclosure but, of course, should not be construed as in any way limiting its scope.

### Example 1

This example demonstrates inhibition of virus, in accordance with aspects of the disclosure.

### Methods

### Cells

A549 expressing H2B-mRuby were generated by first infecting A549 cells (ATCC CCL-185) with a lentivirus (carrying H2B-mRuby), and FACS-sorting mRuby+ cells. They were maintained as a polyclonal population and grown in DMEM+10% BCS (Bovine Calf Serum). These cells were used for all OC43 infections. Ace2-A549 (Blanco-Melo et al., Cell, 181: 1036-1045.e9 (2020), incorporated by reference herein) cells were used for SARS-CoV-2 infections. They were maintained in DMEM+10% FBS. African green monkey kidney cells (Vero E6) were maintained in DMEM supplemented with 10% FBS, 1% penicillin-streptomycin and 1% HEPES. MDCK-SIAT1-TMPRSS2 cells were used for IAV infections. A549 cells maintained in 50:50 DMEM:F-12 media supplemented with 10% FBS and 1% penicillin-streptomycin were used for LCMV infections.

### Viruses

OC43 was obtained from ATCC (VR-1558) grown and titrated on A549-mRuby cells. SARS-CoV-2 (nCoV/Washington/1/2020) was provided by the National Biocontainment Laboratory, Galveston, Tex., USA. VeroE6 cells were used to propagate and titer SARS-CoV-2. CVB3 (Nancy strain), HRV 2, 14, and 16 were derived from full-length infectious clones and generated in Vero cells (NR-10385, BEI Resources, NIAID, NIH). Recombinant LCMV based on the Armstrong 53b strain was generated as previously described (Flatz et al., Proc. Natl. Acad. Sci. U.S.A., 103: 4663-4668 (2006) and Ziegler et al., PLoS Pathog., 12: e1005501 (2016), each of which is incorporated by reference herein). Working stocks were generated in Vero E6 cells, and the same cells were used to measure virus titers. The Measles Virus used was derived from the molecular cDNA clone of the Moraten/Schwartz vaccine strain (del Valle et al., J. Virol., 81: 10597-10605 (2007), incorporated by reference herein). The recombinant Measles virus was engineered to express firefly luciferase as previously described (Muñoz-Alía et al., Viruses, 11: (2019), incorporated by reference herein).

### Drug Screening

A549-mRuby cells were seeded (3,000 cells per well) in nine 384-well plates using Multidrop combi. Cells were seeded in a final volume of 30 μl with DMEM+10% BCS. The following day, 20 μl of OC43 were added (MOI 0.3) and incubated at 33° C., 5% CO2 for 1 hour. 50 nl from the Selleck FDA-approved drug library (cat #L1300, Selleck) were added (1:1,000 dilution). Two columns (32 wells) were left uninfected and two columns were treated with DMSO and virus (no drug control). Cells were imaged using the IncuCyte S3 to measure cell numbers at day 0. Cells were incubated for 4 days at 33° C., 5% CO2 and were stained for OC43 nucleoprotein. All the following steps were performed at room temperature. Cells were fixed in 50 μl 4% PFA/PBS for 15 min, blocked with 50 μl 10% BSA+0.5% Triton X-100 in PBS for 30 minutes, stained with 50 μl anti-OC43 (cat #MAB9013, Milipore) diluted 1:2,000 in 2% BSA+0.1% Triton X-100 in PBS for 1 hour, washed with 50 μl PBS three times, stained with anti-mouse-AlexaFluor488 diluted 1:1,000 in 2% BSA+0.1% Triton X-100 in PBS for 1 hour, washed with 50 μl PBS three times and imaged on the IncuCyte S3 (day 4). The screen was performed twice.

The following parameters were extracted from the images: number of cells at day 0, number of cells at day 4 and total OC43 staining intensity at day 4. For analysis, OC43 staining intensity was normalized to the number of cells in the well and further normalized to the mean of the no drug controls, which was set to 100. Removed from analysis were compounds that showed significant effect on cell growth. For each plate, a drug was considered as a putative hit if it reduced OC43 staining by over 3 standard deviations from the mean of the no drug controls. Drugs were considered hits if they were not toxic and reduced OC43 staining by over 3 standard deviations in both repeats. A list of screened drugs, as well as their effect on cell growth and OC43 staining is summarized in Tables 1A and 1B.

TABLE 1A*

| Drug name | OC43_mean | Plate | Position | index | OC43_1 | OC43_2 |
|---|---|---|---|---|---|---|
| Butamben | 4.9 | 4 | E14 | 213 | 3.8 | 6.1 |
| Butylparaben | 5.2 | 4 | G14 | 215 | 7.0 | 3.5 |
| Conivaptan HCl | 5.7 | 2 | G18 | 27 | 4.5 | 7.0 |
| Amphotericin B | 6.6 | 1 | D12 | 180 | 1.5 | 11.8 |
| Pentoxyverine Citrate | 9.3 | 6 | G5 | 71 | 10.8 | 7.7 |
| Lapatinib | 9.7 | 2 | A18 | 273 | 5.2 | 14.1 |
| Vilazodone HCl | 9.8 | 4 | F13 | 198 | 13.0 | 6.7 |
| Imatinib (STI571) | 10.0 | 2 | H16 | 248 | 10.5 | 9.5 |
| Benztropine mesylate | 10.1 | 3 | C8 | 115 | 7.4 | 12.8 |
| Raloxifene HCl | 11.7 | 1 | B5 | 66 | 9.4 | 14.0 |
| Solifenacin succinate | 12.1 | 7 | C17 | 259 | 13.0 | 11.3 |
| Chloroquine Phosphate | 12.2 | 7 | D5 | 68 | 11.3 | 13.1 |
| Retapamulin | 12.2 | 3 | N18 | 286 | 11.7 | 12.6 |
| Bafetinib (INNO-406) | 13.4 | 1 | J21 | 330 | 17.2 | 9.6 |
| Imipramine HCl | 14.5 | 5 | B18 | 274 | 6.7 | 22.3 |
| Hydroxychloroquine Sulfate | 16.2 | 7 | J7 | 106 | 17.8 | 14.6 |
| Lapatinib ditosylate monohydrate | 16.6 | 5 | K14 | 219 | 23.8 | 9.5 |
| Tolterodine tartrate | 16.6 | 3 | K7 | 107 | 21.7 | 11.6 |
| Clomipramine HCl | 17.7 | 3 | A7 | 97 | 13.9 | 21.6 |
| Velpatasvir | 18.0 | 3 | K22 | 347 | 26.0 | 10.0 |
| Azithromycin Dihydrate | 18.2 | 4 | I13 | 201 | 20.5 | 15.9 |
| Erythromycin Cyclocarbonate | 18.9 | 5 | D3 | 36 | 16.7 | 21.1 |
| Cediranib (AZD2171) | 19.0 | 1 | E5 | 69 | 17.8 | 20.2 |
| Azelastine HCl | 19.1 | 3 | O7 | 111 | 19.6 | 18.5 |
| Desloratadine | 19.3 | 3 | N10 | 158 | 13.2 | 25.4 |
| Nortriptyline hydrochloride | 19.6 | 3 | E20 | 309 | 25.2 | 14.0 |
| Imatinib Mesylate (STI571) | 19.6 | 1 | M5 | 77 | 26.9 | 12.3 |
| Propafenone HCl | 19.8 | 2 | J20 | 314 | 20.4 | 19.2 |
| Mebeverine Hydrochloride | 20.0 | 5 | O7 | 111 | 20.1 | 19.9 |
| Erythromycin estolate | 20.1 | 5 | C20 | 307 | 24.2 | 15.9 |
| Elbasvir | 20.1 | 5 | F8 | 118 | 18.8 | 21.4 |
| Cepharanthine | 20.3 | 6 | K5 | 75 | 28.5 | 12.1 |
| Mesoridazine Besylate | 20.3 | 5 | H12 | 184 | 15.6 | 25.0 |
| Flupenthixol dihydrochloride | 21.0 | 3 | C18 | 275 | 8.7 | 33.3 |
| Eliglustat | 21.5 | 5 | B9 | 130 | 22.7 | 20.4 |
| Cobicistat (GS-9350) | 21.8 | 3 | D7 | 100 | 23.0 | 20.7 |
| Desipramine Hydrochloride | 21.8 | 5 | C8 | 115 | 20.0 | 23.7 |
| Prochlorperazine Dimaleate | 22.6 | 6 | A7 | 97 | 20.2 | 25.0 |
| Saracatinib (AZD0530) | 22.7 | 1 | G3 | 39 | 21.2 | 24.1 |
| Amitriptyline HCl | 22.8 | 3 | I10 | 153 | 11.2 | 34.3 |
| Amiodarone HCl | 22.8 | 2 | L17 | 268 | 18.8 | 26.9 |
| Promazine hydrochloride | 23.0 | 5 | I6 | 89 | 23.4 | 22.6 |
| Nystatin (Fungicidin) | 23.3 | 2 | B13 | 194 | 27.8 | 18.8 |
| Azithromycin | 23.4 | 2 | A21 | 321 | 20.4 | 26.4 |
| Gefitinib hydrochloride | 23.9 | 6 | M11 | 173 | 20.0 | 27.8 |
| Promethazine HCl | 24.4 | 4 | B19 | 290 | 19.4 | 29.5 |
| Carvedilol | 24.6 | 2 | K19 | 299 | 20.1 | 29.1 |
| Darifenacin HBr | 24.7 | 3 | O4 | 63 | 14.6 | 34.8 |
| Propranolol HCl | 24.7 | 3 | P22 | 352 | 21.6 | 27.7 |
| Lapatinib (GW-572016) Ditosylate | 24.8 | 1 | O5 | 79 | 17.6 | 32.0 |
| Solifenacin | 25.0 | 5 | I14 | 217 | 20.4 | 29.6 |
| Cisapride | 25.3 | 5 | A6 | 81 | 28.5 | 22.1 |
| Pizotifen Malate | 25.4 | 1 | K6 | 91 | 33.3 | 17.5 |
| Ifenprodil Tartrate | 25.6 | 4 | K5 | 75 | 29.0 | 22.1 |
| Sarpogrelate hydrochloride | 25.9 | 3 | I20 | 313 | 26.8 | 24.9 |
| Benzydamine HCl | 25.9 | 4 | O15 | 239 | 26.2 | 25.6 |
| Maprotiline HCl | 26.0 | 3 | E3 | 37 | 27.0 | 25.1 |
| Enasidenib (AG-221) | 26.1 | 5 | L15 | 236 | 23.3 | 29.0 |
| Betrixaban maleate | 26.9 | 6 | E21 | 325 | 32.0 | 21.8 |
| Cyproheptadine HCl | 27.0 | 2 | I6 | 89 | 22.8 | 31.2 |
| Propiverine hydrochloride | 27.3 | 5 | E3 | 37 | 29.5 | 25.2 |
| Alverine Citrate | 28.7 | 3 | H15 | 232 | 22.1 | 35.3 |
| Budesonide | 28.8 | 1 | B13 | 194 | 20.8 | 36.9 |
| Carvedilol Phosphate | 28.9 | 5 | M22 | 349 | 32.3 | 25.5 |
| Tepotinib (EMD 1214063) | 28.9 | 5 | I15 | 233 | 15.3 | 42.6 |
| Doxazosin Mesylate | 29.7 | 1 | L15 | 236 | 50.1 | 9.2 |
| Salmeterol Xinafoate | 29.9 | 4 | H19 | 296 | 39.3 | 20.4 |
| Fluvoxamine maleate | 30.8 | 1 | N17 | 270 | 22.7 | 38.9 |
| Clozapine | 31.3 | 2 | H14 | 216 | 15.8 | 46.9 |
| Elacridar (GF120918) | 31.4 | 5 | L5 | 76 | 24.4 | 38.3 |
| Trifluoperazine 2HCl | 31.6 | 3 | M12 | 189 | 33.8 | 29.5 |
| Betrixaban | 32.0 | 5 | I10 | 153 | 30.2 | 33.7 |
| Citalopram HBr | 32.4 | 4 | B16 | 242 | 20.4 | 44.4 |
| Valnemulin HCl | 32.4 | 4 | J5 | 74 | 27.7 | 37.1 |
| Dalbavancin | 32.5 | 4 | J20 | 314 | 25.7 | 39.2 |
| Doxepin HCl | 32.6 | 3 | J21 | 330 | 29.2 | 36.1 |
| Dimemorfan phosphate | 33.0 | 7 | P15 | 240 | 39.7 | 26.2 |
| Beclomethasone dipropionate | 33.6 | 3 | B19 | 290 | 38.3 | 29.0 |
| Guanabenz Acetate | 34.6 | 3 | P20 | 320 | 31.1 | 38.1 |
| Gefitinib (ZD1839) | 35.5 | 1 | K5 | 75 | 39.7 | 31.3 |
| Quinidine sulfate | 36.5 | 3 | P6 | 96 | 25.1 | 47.9 |
| Orphenadrine Hydrochloride | 37.6 | 6 | F5 | 70 | 11.7 | 63.6 |
| Anamorelin | 38.8 | 6 | M7 | 109 | 32.0 | 45.6 |
| Lomerizine 2HCl | 39.9 | 4 | O3 | 47 | 56.5 | 23.3 |
| Prochlorperazine dimaleate salt | 40.2 | 4 | A22 | 337 | 47.2 | 33.1 |
| Alimemazine Tartrate | 40.8 | 5 | D4 | 52 | 47.9 | 33.7 |
| Rifabutin | 41.1 | 2 | M7 | 109 | 25.8 | 56.4 |
| Roxithromycin | 41.4 | 2 | F22 | 342 | 55.8 | 27.1 |
| Cinchocaine | 41.6 | 6 | I19 | 297 | 18.9 | 64.3 |
| Dabrafenib (GSK2118436) | 41.6 | 3 | B5 | 66 | 36.3 | 47.0 |
| Diphenylpyraline HCl | 42.4 | 5 | N10 | 158 | 33.2 | 51.6 |
| Perphenazine | 42.8 | 4 | H14 | 216 | 59.8 | 25.8 |
| Diperodon HCl | 42.9 | 5 | B20 | 306 | 24.2 | 61.6 |
| Etonogestrel | 44.3 | 4 | B10 | 146 | 47.8 | 40.7 |
| Fluocinolone Acetonide | 45.5 | 2 | B16 | 242 | 38.9 | 52.2 |
| Cyclobenzaprine HCl | 46.5 | 4 | P15 | 240 | 45.2 | 47.7 |
| Quetiapine Fumarate | 48.0 | 2 | E11 | 165 | 34.5 | 61.5 |
| Fluticasone propionate | 48.8 | 2 | F19 | 294 | 48.4 | 49.2 |
| Propylthiouracil | 49.7 | 2 | B19 | 290 | 44.3 | 55.1 |
| Xylometazoline HCl | 49.7 | 3 | M11 | 173 | 54.0 | 45.4 |
| Vorapaxar | 50.9 | 5 | J13 | 202 | 53.5 | 48.3 |
| Rimantadine Hydrochloride | 52.6 | 5 | A8 | 113 | 50.6 | 54.6 |
| Ibutilide Fumarate | 54.4 | 2 | I18 | 281 | 45.9 | 63.0 |
| Tropisetron HCl | 58.0 | 2 | L7 | 108 | 52.6 | 63.4 |
| Tofacitinib (CP-690550) Citrate | 58.5 | 5 | A5 | 65 | 56.5 | 60.4 |
| Erythromycin thiocyanate | 58.7 | 6 | A13 | 193 | 50.6 | 66.8 |

TABLE 1B*

| Drug name | Growth_1 | Growth_2 | hit_1 | hit_2 | toxic_1 | toxic_2 |
|---|---|---|---|---|---|---|
| Butamben | 3.9 | 4.7 | 1 | 1 | 0 | 0 |
| Butylparaben | 3.3 | 3.6 | 1 | 1 | 0 | 0 |
| Conivaptan HCl | 4.2 | 4.7 | 1 | 1 | 0 | 0 |
| Amphotericin B | 4.3 | 5.8 | 1 | 1 | 0 | 0 |
| Pentoxyverine Citrate | 5.0 | 5.7 | 1 | 1 | 0 | 0 |
| Lapatinib | 3.8 | 4.4 | 1 | 1 | 0 | 0 |
| Vilazodone HCl | 3.5 | 3.9 | 1 | 1 | 0 | 0 |
| Imatinib (STI571) | 4.4 | 4.8 | 1 | 1 | 0 | 0 |
| Benztropine mesylate | 4.4 | 5.1 | 1 | 1 | 0 | 0 |
| Raloxifene HCl | 5.0 | 5.2 | 1 | 1 | 0 | 0 |
| Solifenacin succinate | 5.0 | 5.0 | 1 | 1 | 0 | 0 |
| Chloroquine Phosphate | 4.7 | 4.4 | 1 | 1 | 0 | 0 |
| Retapamulin | 4.3 | 4.1 | 1 | 1 | 0 | 0 |
| Bafetinib (INNO-406) | 5.0 | 3.6 | 1 | 1 | 0 | 0 |
| Imipramine HCl | 4.0 | 4.2 | 1 | 1 | 0 | 0 |
| Hydroxychloroquine Sulfate | 5.1 | 4.9 | 1 | 1 | 0 | 0 |
| Lapatinib ditosylate monohydrate | 4.2 | 4.2 | 1 | 1 | 0 | 0 |
| Tolterodine tartrate | 5.0 | 5.1 | 1 | 1 | 0 | 0 |
| Clomipramine HCl | 2.6 | 3.8 | 1 | 1 | 0 | 0 |
| Velpatasvir | 6.2 | 5.6 | 1 | 1 | 0 | 0 |
| Azithromycin Dihydrate | 5.4 | 5.0 | 1 | 1 | 0 | 0 |
| Erythromycin Cyclocarbonate | 5.7 | 6.0 | 1 | 1 | 0 | 0 |
| Cediranib (AZD2171) | 2.6 | 2.4 | 1 | 1 | 0 | 0 |
| Azelastine HCl | 4.1 | 3.2 | 1 | 1 | 0 | 0 |
| Desloratadine | 4.8 | 4.4 | 1 | 1 | 0 | 0 |
| Nortriptyline hydrochloride | 4.2 | 3.1 | 1 | 1 | 0 | 0 |
| Imatinib Mesylate (STI571) | 5.3 | 5.4 | 1 | 1 | 0 | 0 |
| Propafenone HCl | 4.7 | 3.4 | 1 | 1 | 0 | 0 |
| Mebeverine Hydrochloride | 5.2 | 3.9 | 1 | 1 | 0 | 0 |
| Erythromycin estolate | 4.5 | 4.8 | 1 | 1 | 0 | 0 |
| Elbasvir | 4.7 | 5.3 | 1 | 1 | 0 | 0 |
| Cepharanthine | 4.3 | 4.7 | 1 | 1 | 0 | 0 |
| Mesoridazine Besylate | 4.7 | 4.7 | 1 | 1 | 0 | 0 |
| Flupenthixol dihydrochloride | 2.1 | 3.3 | 1 | 1 | 0 | 0 |
| Eliglustat | 5.5 | 4.8 | 1 | 1 | 0 | 0 |
| Cobicistat (GS-9350) | 4.9 | 5.3 | 1 | 1 | 0 | 0 |
| Desipramine Hydrochloride | 4.4 | 3.9 | 1 | 1 | 0 | 0 |
| Prochlorperazine Dimaleate | 3.1 | 3.0 | 1 | 1 | 0 | 0 |
| Saracatinib (AZD0530) | 3.1 | 4.0 | 1 | 1 | 0 | 0 |
| Amitriptyline HCl | 3.7 | 4.1 | 1 | 1 | 0 | 0 |
| Amiodarone HCl | 4.6 | 5.4 | 1 | 1 | 0 | 0 |
| Promazine hydrochloride | 4.7 | 4.7 | 1 | 1 | 0 | 0 |
| Nystatin (Fungicidin) | 4.6 | 5.9 | 1 | 1 | 0 | 0 |
| Azithromycin | 4.0 | 4.4 | 1 | 1 | 0 | 0 |
| Gefitinib hydrochloride | 3.5 | 3.3 | 1 | 1 | 0 | 0 |
| Promethazine HCl | 3.0 | 2.4 | 1 | 1 | 0 | 0 |
| Carvedilol | 4.5 | 3.1 | 1 | 1 | 0 | 0 |
| Darifenacin HBr | 5.2 | 3.4 | 1 | 1 | 0 | 0 |
| Propranolol HCl | 5.6 | 4.1 | 1 | 1 | 0 | 0 |
| Lapatinib (GW-572016) Ditosylate | 5.0 | 5.9 | 1 | 1 | 0 | 0 |
| Solifenacin | 4.3 | 3.4 | 1 | 1 | 0 | 0 |
| Cisapride | 4.1 | 4.5 | 1 | 1 | 0 | 0 |
| Pizotifen Malate | 3.4 | 2.8 | 1 | 1 | 0 | 0 |
| Ifenprodil Tartrate | 4.6 | 4.9 | 1 | 1 | 0 | 0 |
| Sarpogrelate hydrochloride | 5.0 | 5.4 | 1 | 1 | 0 | 0 |
| Benzydamine HCl | 4.9 | 4.6 | 1 | 1 | 0 | 0 |
| Maprotiline HCl | 3.5 | 3.1 | 1 | 1 | 0 | 0 |
| Enasidenib (AG-221) | 5.2 | 4.8 | 1 | 1 | 0 | 0 |
| Betrixaban maleate | 4.6 | 5.7 | 1 | 1 | 0 | 0 |
| Cyproheptadine HCl | 3.8 | 3.0 | 1 | 1 | 0 | 0 |
| Propiverine hydrochloride | 3.0 | 2.2 | 1 | 1 | 0 | 0 |
| Alverine Citrate | 3.7 | 4.0 | 1 | 1 | 0 | 0 |
| Budesonide | 3.9 | 4.0 | 1 | 1 | 0 | 0 |
| Carvedilol Phosphate | 4.4 | 2.9 | 1 | 1 | 0 | 0 |
| Tepotinib (EMD 1214063) | 5.3 | 5.2 | 1 | 1 | 0 | 0 |
| Doxazosin Mesylate | 5.2 | 4.9 | 1 | 1 | 0 | 0 |
| Salmeterol Xinafoate | 2.9 | 2.5 | 1 | 1 | 0 | 0 |
| Fluvoxamine maleate | 5.4 | 4.5 | 1 | 1 | 0 | 0 |
| Clozapine | 4.6 | 4.3 | 1 | 1 | 0 | 0 |
| Elacridar (GF120918) | 4.6 | 5.8 | 1 | 1 | 0 | 0 |
| Trifluoperazine 2HCl | 2.3 | 2.1 | 1 | 1 | 0 | 0 |
| Betrixaban | 5.3 | 5.5 | 1 | 1 | 0 | 0 |
| Citalopram HBr | 5.1 | 5.1 | 1 | 1 | 0 | 0 |
| Valnemulin HCl | 4.4 | 4.5 | 1 | 1 | 0 | 0 |
| Dalbavancin | 5.8 | 6.2 | 1 | 1 | 0 | 0 |
| Doxepin HCl | 4.9 | 5.7 | 1 | 1 | 0 | 0 |
| Dimemorfan phosphate | 6.0 | 5.4 | 1 | 1 | 0 | 0 |
| Beclomethasone dipropionate | 3.4 | 4.6 | 1 | 1 | 0 | 0 |
| Guanabenz Acetate | 5.3 | 4.4 | 1 | 1 | 0 | 0 |
| Gefitinib (ZD1839) | 3.4 | 3.7 | 1 | 1 | 0 | 0 |
| Quinidine sulfate | 5.6 | 4.1 | 1 | 1 | 0 | 0 |
| Orphenadrine Hydrochloride | 4.6 | 5.1 | 1 | 1 | 0 | 0 |
| Anamorelin | 5.2 | 5.5 | 1 | 1 | 0 | 0 |
| Lomerizine 2HCl | 4.7 | 5.3 | 1 | 1 | 0 | 0 |
| Prochlorperazine dimaleate salt | 3.1 | 3.1 | 1 | 1 | 0 | 0 |
| Alimemazine Tartrate | 2.6 | 2.2 | 1 | 1 | 0 | 0 |
| Rifabutin | 5.5 | 5.3 | 1 | 1 | 0 | 0 |
| Roxithromycin | 5.2 | 5.6 | 1 | 1 | 0 | 0 |
| Cinchocaine | 4.3 | 4.2 | 1 | 1 | 0 | 0 |
| Dabrafenib (GSK2118436) | 3.9 | 5.5 | 1 | 1 | 0 | 0 |
| Diphenylpyraline HCl | 6.0 | 4.2 | 1 | 1 | 0 | 0 |
| Perphenazine | 2.2 | 2.5 | 1 | 1 | 0 | 0 |
| Diperodon HCl | 4.6 | 4.4 | 1 | 1 | 0 | 0 |
| Etonogestrel | 4.3 | 4.3 | 1 | 1 | 0 | 0 |
| Fluocinolone Acetonide | 3.5 | 4.0 | 1 | 1 | 0 | 0 |
| Cyclobenzaprine HCl | 3.3 | 2.1 | 1 | 1 | 0 | 0 |
| Quetiapine Fumarate | 3.6 | 3.7 | 1 | 1 | 0 | 0 |
| Fluticasone propionate | 3.8 | 4.2 | 1 | 1 | 0 | 0 |
| Propylthiouracil | 3.7 | 4.2 | 1 | 1 | 0 | 0 |
| Xylometazoline HCl | 5.4 | 5.7 | 1 | 1 | 0 | 0 |
| Vorapaxar | 4.4 | 4.1 | 1 | 1 | 0 | 0 |
| Rimantadine Hydrochloride | 4.4 | 5.1 | 1 | 1 | 0 | 0 |
| Ibutilide Fumarate | 4.8 | 4.8 | 1 | 1 | 0 | 0 |
| Tropisetron HCl | 5.8 | 5.5 | 1 | 1 | 0 | 0 |
| Tofacitinib (CP-690550) Citrate | 4.8 | 4.7 | 1 | 1 | 0 | 0 |
| Erythromycin thiocyanate | 4.1 | 4.6 | 1 | 1 | 0 | 0 |

key for Tables 1A and 1B:
Plate: number of plate in the screen (drugs were divided into 9 plates)
index: well index (1-384)
position: well position (rows A-P, columns 1-24)
OC43_1: % staining of OC43 in screen repeat #1 (normalized to no-drug controls in the same plate)
OC43_2: % staining of OC43 in screen repeat #2 (normalized to no-drug controls in the same plate)
Growth_1: number of cells at day 4 divided by number of cells at day 0 (repeat #1)
Growth_2: number of cells at day 4 divided by number of cells at day 0 (repeat #2)
Hit_1: 0=not a hit, 1=is a hit (OC43 staining is less than 3 standard deviations from the mean of no drug controls), repeat #1
Hit_2: 0=not a hit, 1=is a hit (OC43 staining is less than 3 standard deviations from the mean of no drug controls), repeat #2
toxic_1: 0=not toxic, 1—toxic (cells number at day 4 is less than double the number at day 0), repeat #1
toxic_2: 0=not toxic, 1—toxic (cells number at day 4 is less than double the number at day 0), repeat #2
mock/no drug: mock=uninfected control, no drug=no drug control, otherwise N/A
Drug name: name of the drug added
Hits: data for drugs that were designated hits in the two repeats and did not show significant toxicity

### Dose-Response Analysis for OC43 and SARS-CoV-2 Infection

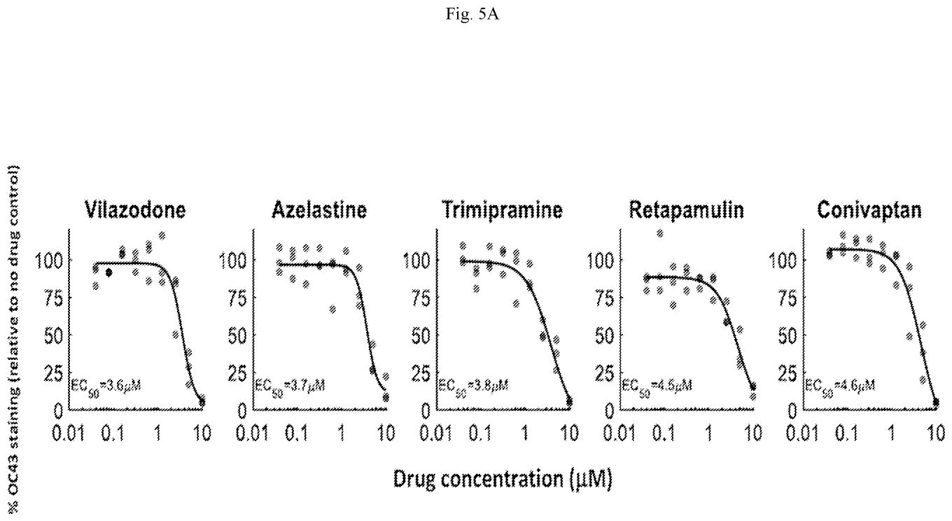

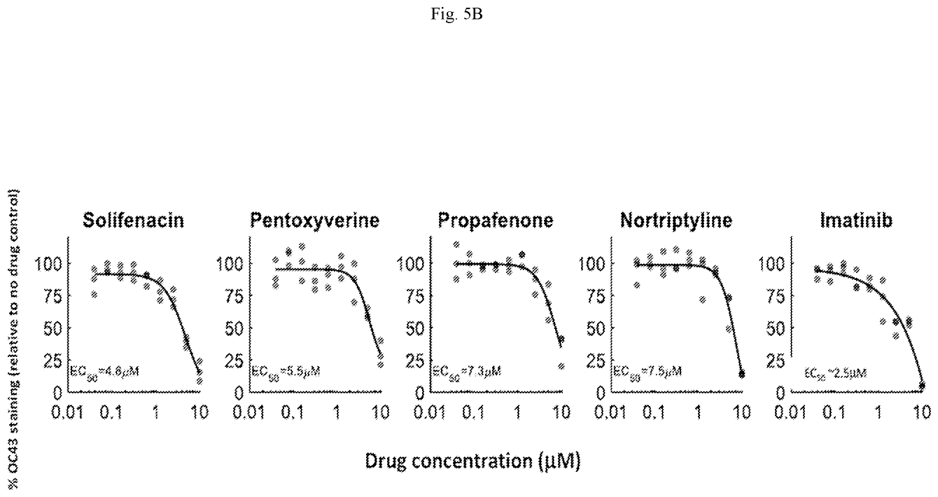

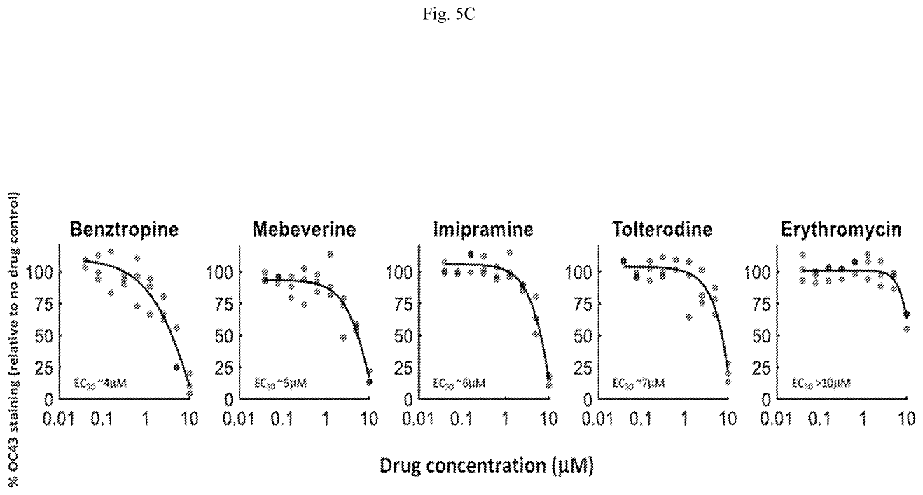

Dose-response analysis of OC43 infection was done similarly to the drug screening, except cells were seeded at a concentration of 5,000 cells per well and the media contained 2% BCS instead of 10% BCS. OC43 staining was performed 2 days after infection and analyzed similarly to what was described for the drug screening. A sigmoid fit was used to extract EC50 values using Matlab. In a few cases a sigmoid curve could not be fit to the data and EC50 values were estimated from the graphs (these are denoted by ˜ in FIGS. 5A-5C).

All SARS-CoV-2 infections were performed in biosafety level 3 conditions at the Howard T. Ricketts Regional Biocontainment Laboratory. Ace2-A549 cells in DMEM +2% FBS were treated with drugs for 2 hours with 2-fold dilutions beginning at 10 μM in triplicate for each assay. Cells were infected with an MOI of 0.5 in media containing the appropriate concentration of drugs. After 48 hours, the cells were fixed using 3.7% formalin, blocked and probed with mouse anti-Spike antibody (GTX632604, GeneTex) diluted 1:1,000 for 4 hours, rinsed and probed with anti-mouse-HRP for 1 hour, washed, then developed with DAB substrate 10 minutes. Spike positive cells (n>40) were quantified by light microscopy as blinded samples.

For SARS-CoV-2 plaque titers, cell supernatants from the infection described above were serially diluted (10-fold steps were used) and used to infect Vero E6 cells for 1 hour. Inoculum was removed and 1.25% methylcellulose DMEM solution was added to the cells and incubated for 3 days. Plates were fixed in 1:10 formalin for 1 hour, stained with crystal violet for 1 hour and were counted to determine plaque forming units (PFU)/ml.

### FlipGFP SARS-CoV-2 3CLpro Assay

293T cells were seeded 24 hours before transfection on poly-lysine treated plates. The next day, SARS-CoV-2 3CLpro plasmid, FlipGFP coronavirus reporter plasmid, Opti-MEM, and TransIT-LT (Mirus) were combined and incubated at room temperature for 20 minutes before being added to the cells. Drugs were applied to the cells at the indicated concentrations at the time of transfection. 24 hours after transfection, cells were fixed with 2% PFA at room temperature for 20 minutes and were incubated in 1:10,000 Hoescht 33342 (Life Technologies) in PBS at 4° C. overnight. Quantification was performed by using the CellInsight CX5 (Thermo Scientific) equipment.

### Gene Cloning, Protein Over-Expression and Purification

Cloning of 3CLpro from SARS CoV-2 was based upon the original cloning of SARS-CoV 3CLpro (Xue et al., J. Mol. Biol., 366: 965-975 (2007), incorporated by reference herein). The gene coding for 3CLpro from SARS CoV-2 was cloned between an upstream MBP and a downstream sequence of GPHHHHHH (SEQ ID NO: 1). Detailed cloning of pCSGID-Mpro carrying 3CLpro from SARS CoV-2 is described in Kneller et al. (Kneller et al., Nat. Commun., 11: 3202 (2020), incorporated by reference herein).

pCSGID-Mpro was transformed into 100 ml of E. coli BL21(DE3)-Gold (Strategene) under selection of ampicillin (150 mg/L) and grown overnight at 37° C. The starter was then transferred to 4 L of LB-Miller, culture and was grown at 37° C. with constant shaking (190 rpm). After reaching an OD600 of ˜1, the shaker was set to 4° C. When temperature reached 18° C., IPTG and K₂HPO₄ was added to 0.2 mM and 40 mM respectively and the culture was marinated at 18° C. The cells were spun down at 4000 g, resuspended in lysis buffer (500 mM NaCl, 5% (v/v) glycerol, 50 mM HEPES pH 8.0, 20 mM imidazole pH 8.0, 1 mM TCEP) and kept frozen at −80° C.

Bacterial cells were lysed by sonication and debris were removed by centrifugation at 25,400×g for 60 min at 4° C. The clarified supernatant was mixed with 3 ml of Ni²⁺ Sepharose (GE Healthcare Life Sciences) equilibrated with lysis buffer. The suspension was applied to a Flex-Column (420400-2510) which was connected to a Vac-Man vacuum manifold. Unbound protein was washed out using controlled suction lysis buffer (160 ml). 3CLpro was eluted using 15 ml of buffer containing 500 mM NaCl, 5% (v/v) glycerol, 50 mM HEPES pH 8.0, 500 mM imidazole pH 8.0 and 1 mM TCEP. The fractions containing 3CLpro were pooled, and rhinovirus 3C His6 tagged protease was added at a 1:25 protease:protein ratio and incubated at 4° C. overnight to cleave the C-terminal His6 tag, resulting in a 3CLpro with an authentic N and C-termini. 10 kDa MWCO filter (Amicon-Millipore) was used to concentrate the protein solution, which was subsequently applied to Superdex 75 column, pre-equilibrated with lysis buffer. The fractions containing 3CLpro were pooled together and run through 2 ml of Ni resin. The flow through was collected and the lysis buffer was replaced with crystallization buffer (20 mM HEPES pH 7.5, 150 mM NaCl, 2 mM DTT (1,4-Dithiothreitol, Roche, Basel, Switzerland)) using a 10 kDa MWCO filter. 3CLpro solution was concentrated to 49 mg/ml, was aliquoted, frozen and stored at −80° C.

### 3C Protease Activity Assay

Huh7 cells were transfected with LipoD293 (SignaGen Laboratories) with 3C substrate, 3C protease (derived from CVV3) and a Renilla transfection control plasmid (siCheck). Protease and target constructs were generated using protocols previously described (Dial et al., Viruses 11(5):403, (2019), incorporated by reference). The cells were combined with firefly substrate (Bright-Glo; Promega) followed by subsequent Renilla (Stop and Glo; Promega) luciferase substrate 24 hours post transfection. Assays were performed using the manufacturer's recommendations (Promega) and a Veritas Microplate Luminometer (Turner BioSystems) was used to quantify the results.

### Statistical Analyses

For all experiment described, the size of the sample (n) refers to independent biological samples tested. All analyses were performed in Matlab. Multiple-comparison corrections was performed using the FDR method.

### Results

A drug repurposing screen against the human beta coronavirus OC43 identifies multiple drugs that are effective against SARS-CoV-2

A library of 1,900 clinically used drugs was screened, the drugs either approved for human use or having extensive safety data in humans (Phase 2 or 3 clinical trials), for their ability to inhibit OC43 infection of the human lung epithelial cell line A549 (expressing an H2B-mRuby nuclear reporter). One day after plating, cells were infected at an MOI of 0.3, incubated at 33° C. for 1 hour and drugs were added to a final concentration of 10 μM. Cells were then incubated at 33° C. for 4 days, fixed and stained for the presence of the viral nucleoprotein. The cells were imaged at day 0 (following drug addition) and day 4 (after staining) to determine the drugs effect on cell growth and OC43 infection.

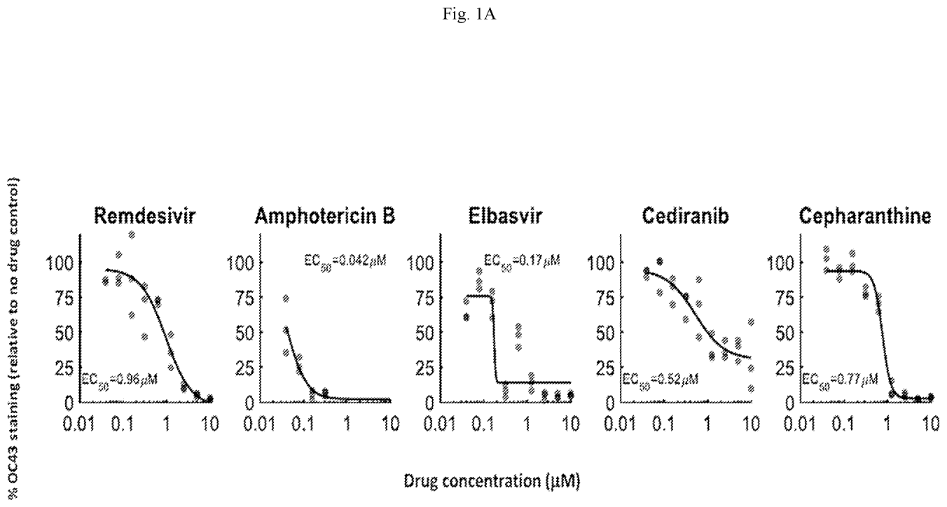

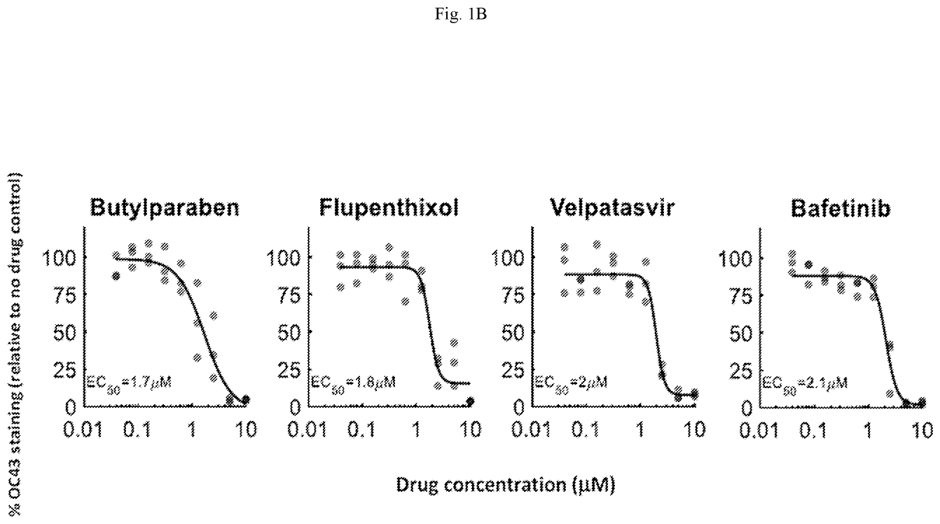

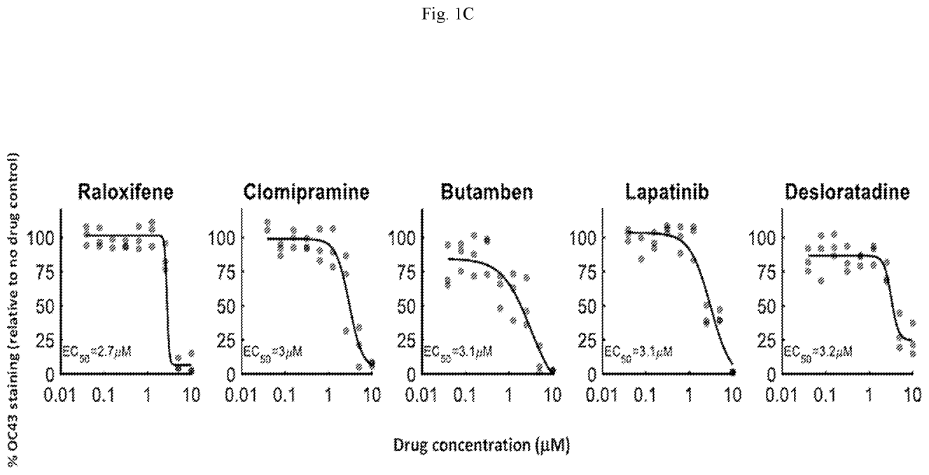

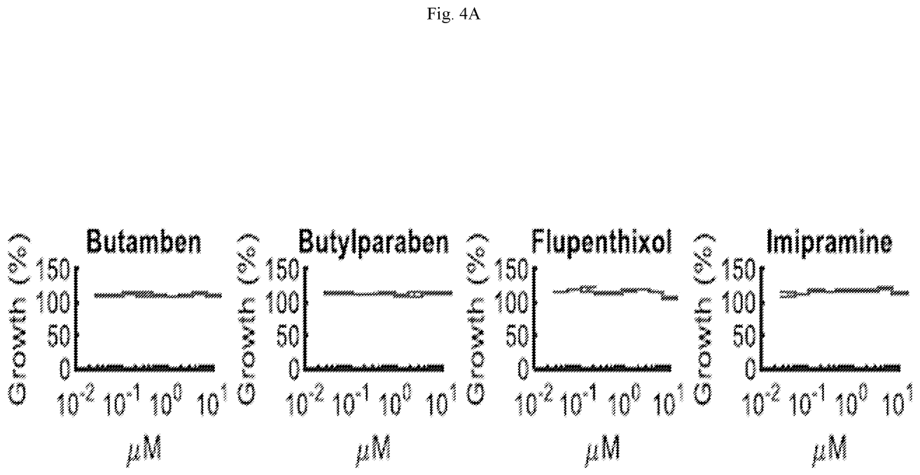

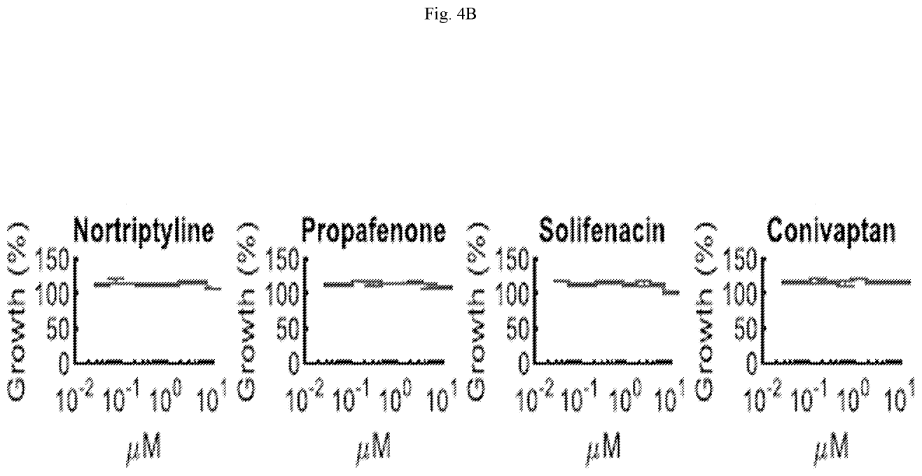

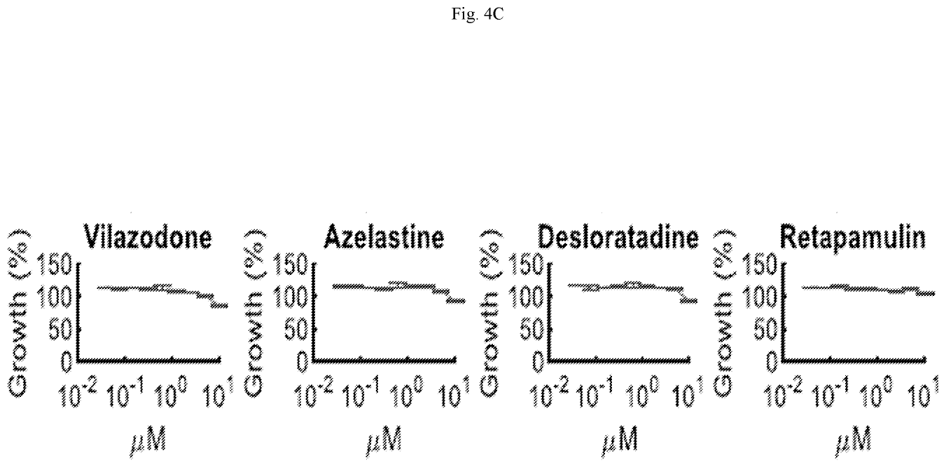

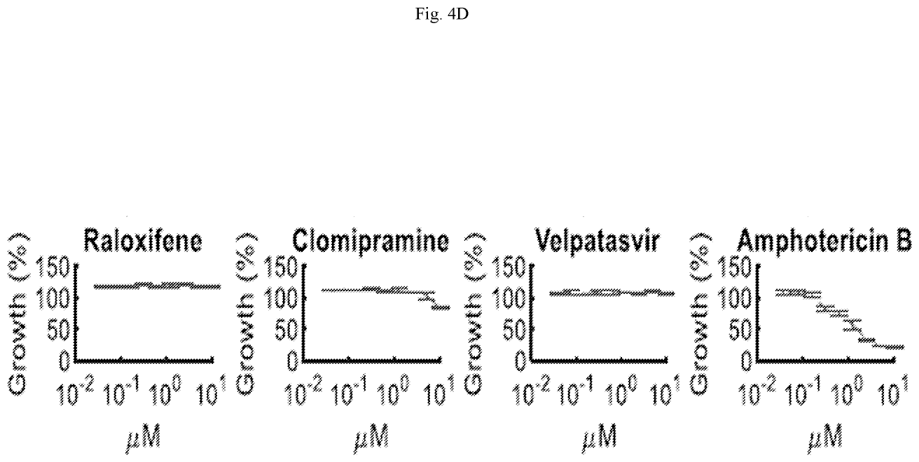

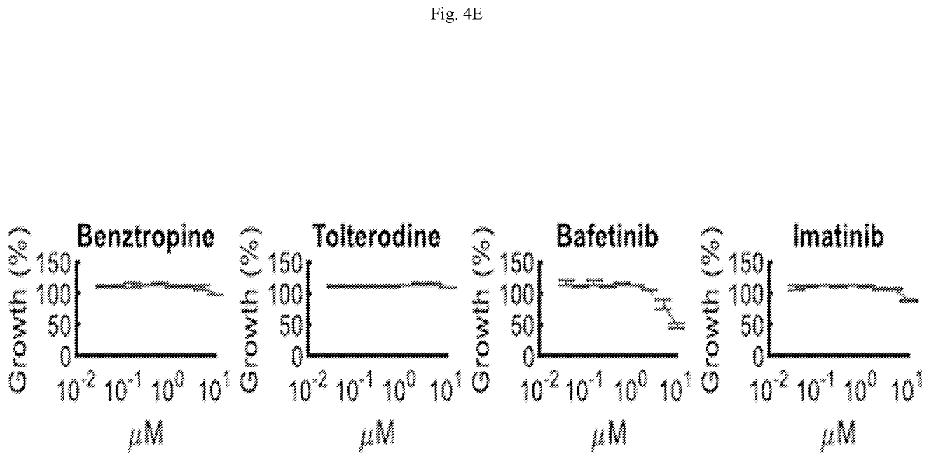

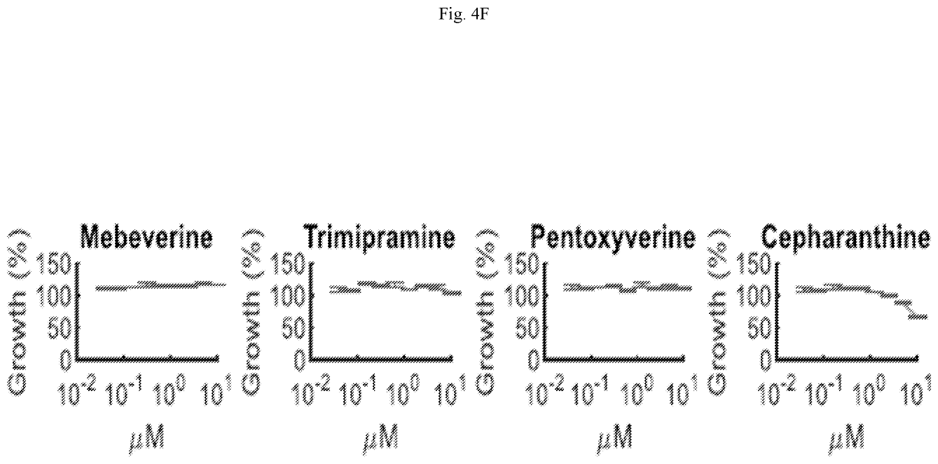

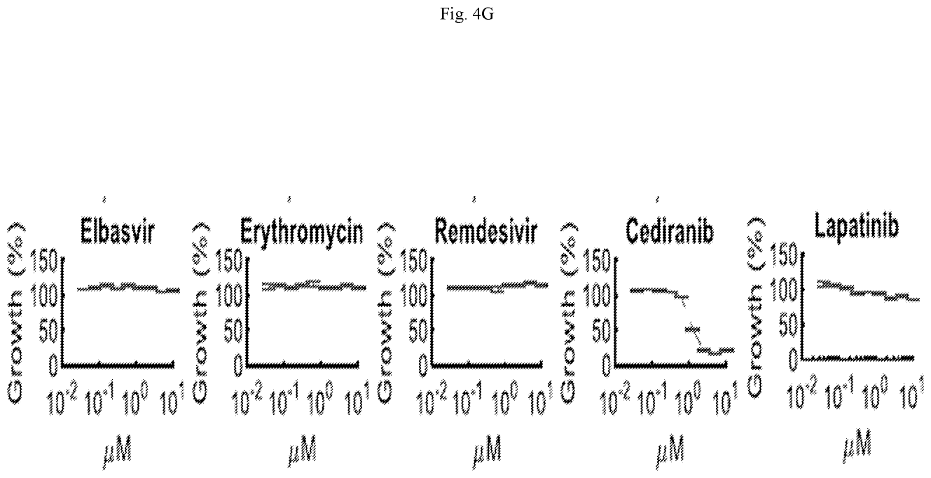

The screen was repeated twice and identified 108 drugs that significantly reduced OC43 infection without significant cellular toxicity (FIGS. 4A-4G, Tables 1A and 1B). Overall agreement between the two repeats was high (R2=0.81). Of the top hits, 29 drugs were reselected for further validation. The EC50 values (drug concentration required to reduce infection by 50%) were determined for these drugs against OC43 infection (FIGS. 1A-1C, FIGS. 5A-5C). All of the drugs except erythromycin inhibited OC43 infection in a dose-dependent manner, with EC50 values ranging from 0.04-7 μM. The most potent drugs in inhibiting OC43 infection were amphotericin B (an anti-fungal drug, EC50=0.04 μM), elbasvir (an anti-viral against HCV, EC50=0.17 μM), cediranib (an EGFR inhibitor, EC50=0.52 μM) and cepharanthine (an anti-inflammatory drug, EC50=0.77 μM). Remdesivir inhibited OC43 infection in the same cells with an EC50 value of 0.9 μM.

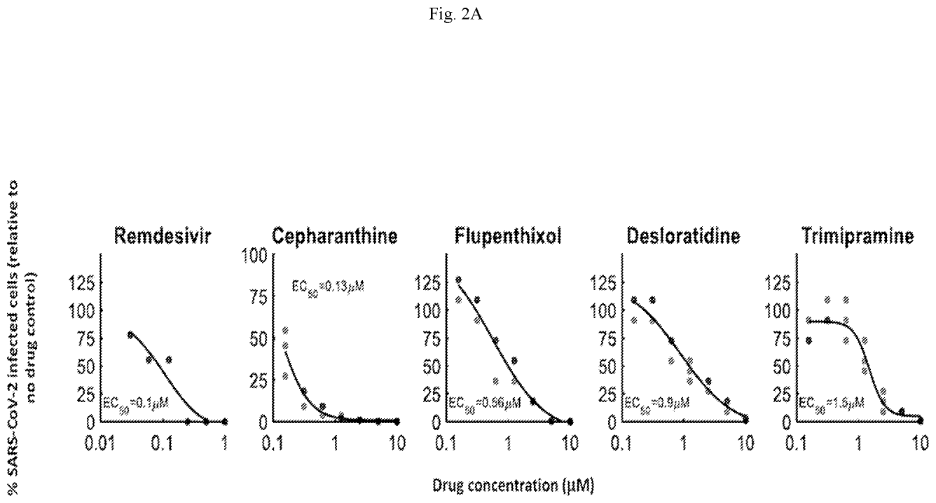

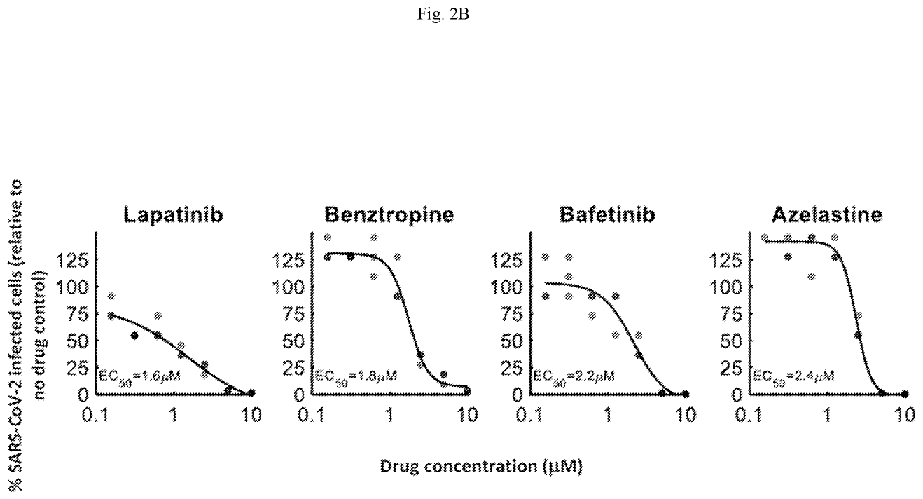

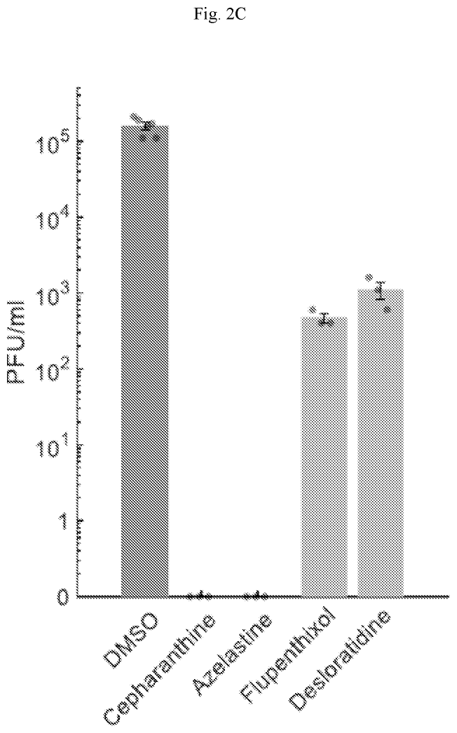

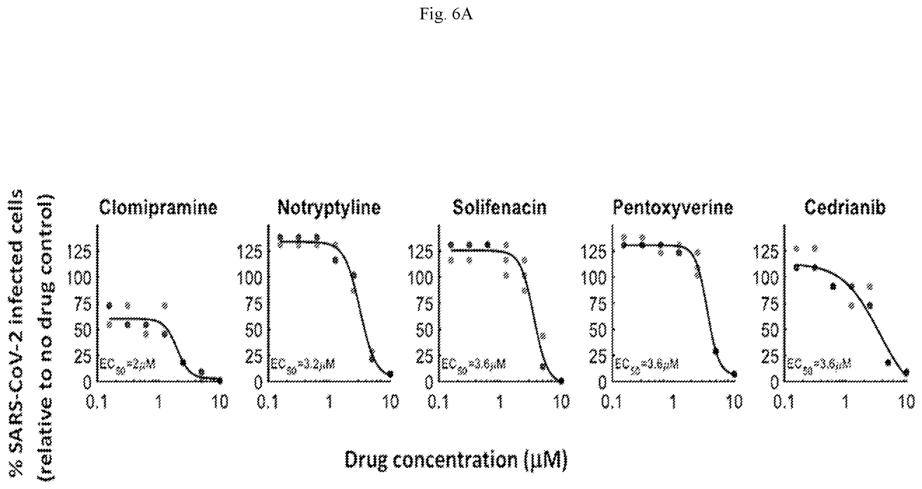

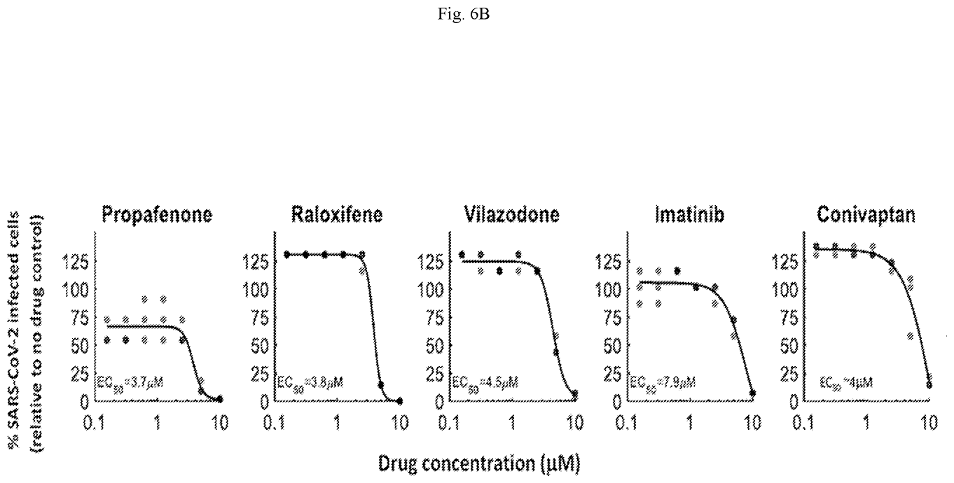

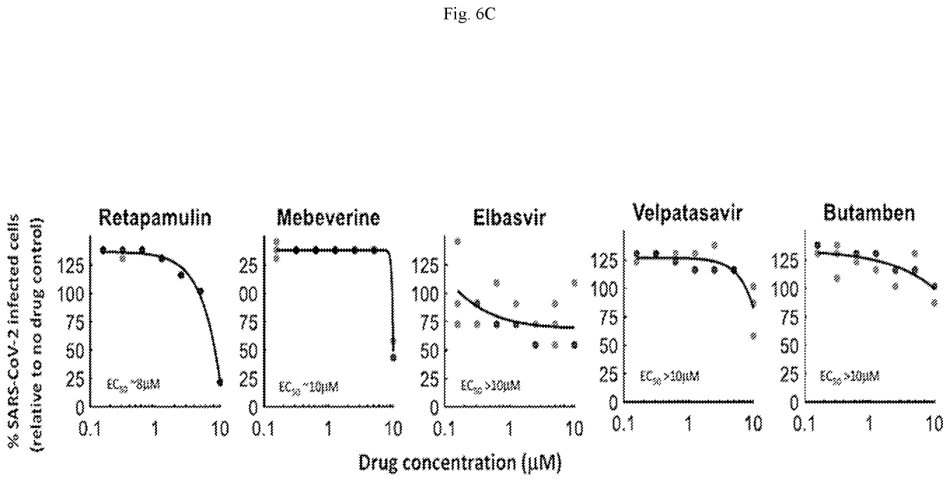

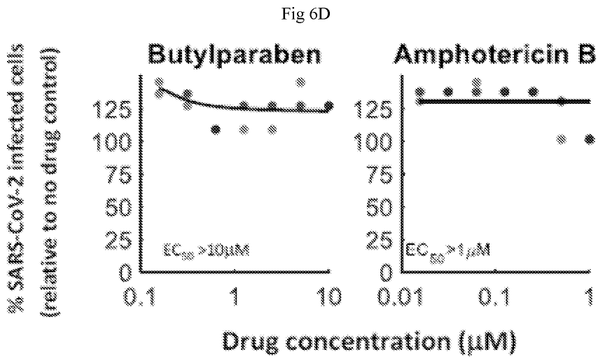

The EC50 values against SARS-CoV-2 infection for 26 of these drugs were determined. In a high biocontainment (BSL3) facility, A549 cells over-expressing the angiotensin-converting enzyme 2 (ACE2) receptor were treated with the drugs for 2 hours, infected with SARS-CoV-2 at an MOI of 0.5, incubated for 2 days, fixed, and stained for the viral spike protein (as a marker of SARS-CoV-2 infection). After staining, the cells were imaged under a microscope to quantify the fraction of infected cells. Of the 26 drugs tested, 20 (77%) inhibited SARS-CoV-2 infection in a dose dependent manner (FIGS. 2A and 2B, FIGS. 6A-6D). The most potent drugs against OC43 infection (amphotericin B and elbavir) did not inhibit SARS-CoV-2 infection. The pleiotropic drug cepharanthine was the most potent inhibitor of SARS-CoV-2 infection (EC50=0.13 μM), followed by flupenthixol (an anti-psychotic, EC50=0.56 μM) and desloratidine (an anti-histamine, EC50=0.9 μM). In comparison, remdesivir inhibited SARS-CoV-2 infection with an EC50 value of 0.1 μM. A comparison of the EC50 values obtained against OC43 and SARS-CoV-2, as well as the chemical structures of the drugs, is shown in the table below.

TABLE 3

| | | EC50 | EC50 | |
|---|---|---|---|---|
| # | Drug | OC43 | SARS2 | Structure |
| 1 | Butamben (n-butyl-4- aminobenzoate) | 3.1 | >10 | 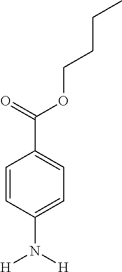 |
| 2 | Butylparaben (n-buytl- 4-hyfroxybenzoate) | 1.7 | >10 | 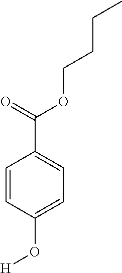 |
| 3 | Flupenthixol dihydrochloride | 1.8 | 0.56 | 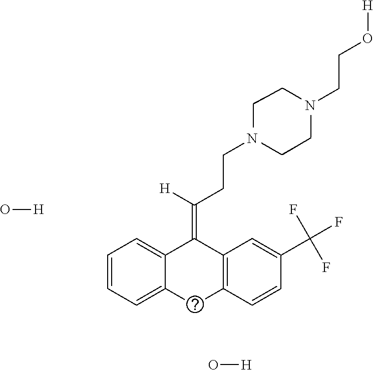 |
| 4 | Imipramine HCl | 6 | ND | 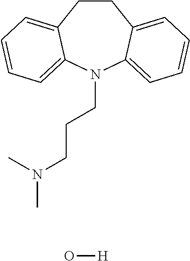 [SMILES: CN(C)CCCN1c2ccccc2CCc2ccccc21 ; O] |
| 5 | Nortriptyline hydrochloride | 7.5 | 3.2 | 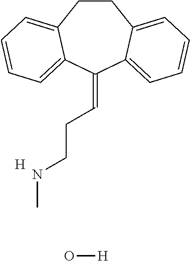 |
| 6 | Propafenone HCl | 7.3 | 3.7 | 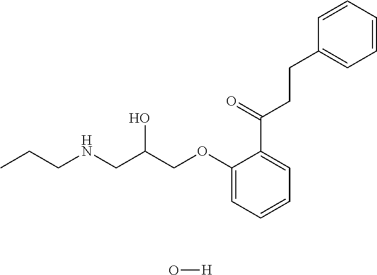 [SMILES: CCCNCC(O)COc1ccccc1C(=O)CCc1ccccc1 ; O] |
| 7 | Solifenacin succinate | 4.8 | 3.6 | 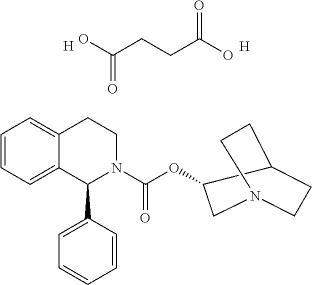 |
| 8 | Conivaptan HCl | 4.6 | 4 | 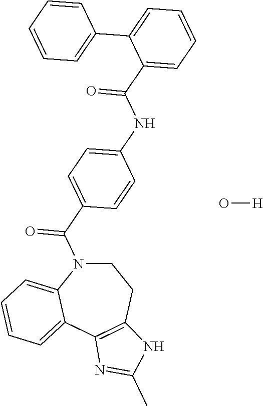 [SMILES: Cc1nc2c([nH]1)CCN(C(=O)c1ccc(NC(=O)c3ccccc3-c3ccccc3)cc1)c1ccccc1-2 ; O] |
| 9 | Vilazodone HCl | 3.6 | 4.5 | 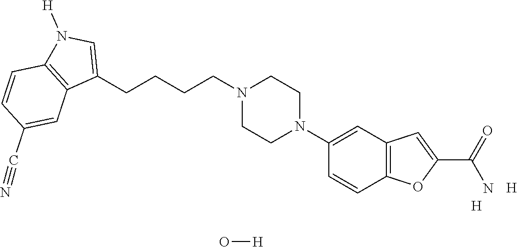 |
| 10 | Azelastine HCl | 3.7 | 2.4 | 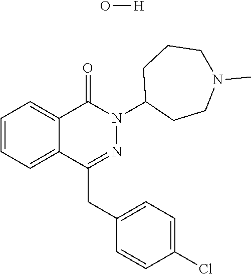 [SMILES: O ; CN1CCCC(n2nc(Cc3ccc(Cl)cc3)c3ccccc3c2=O)CC1] |
| 11 | Desloratadine | 3.2 | 0.9 | 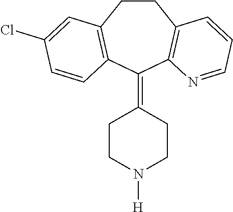 [SMILES: Clc1ccc2c(c1)CCc1cccnc1C2=C1CCNCC1] |
| 12 | Retapamulin | 4.5 | 8 | 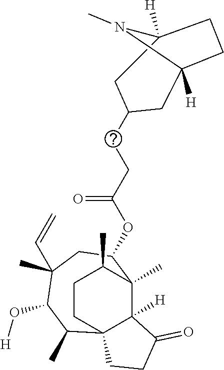 [SMILES: C=C[C@]1(C)C[C@H](OC(=O)CCC2C[C@H]3CC[C@@H](C2)N3C)[C@]2(C)[C@H](C)CC[C@]3(CCC(=O)[C@@H]32)[C@@H](C)[C@@H]1O] |
| 13 | Raloxifene HCl | 2.7 | 3.8 | 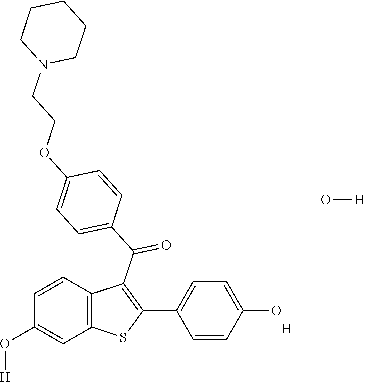 |
| 14 | Clomipramine HCl | 3 | 2 | 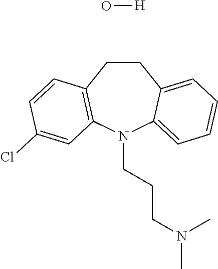 [SMILES: O ; CN(C)CCCN1c2ccccc2CCc2ccc(Cl)cc21] |
| 15 | Velpatasvir | 2 | >10 | 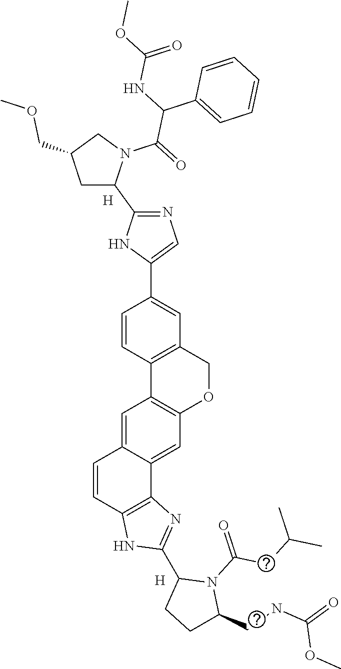 |
| 16 | Amphotericin B | 0.04 | >1 | 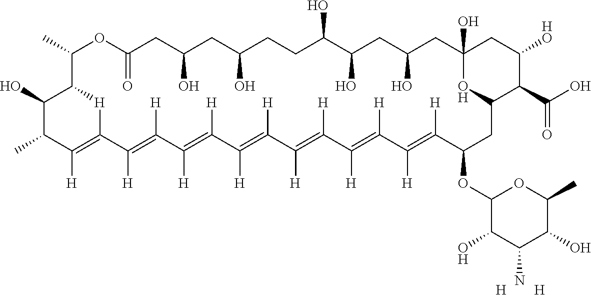 |
| 17 | Benztropine mesylate | 4 | 1.8 | 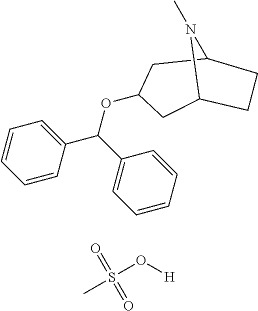 |
| 18 | Tolterodine tartrate | 7 | ND | 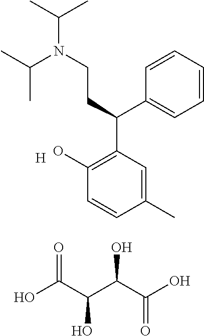 |
| 19 | Bafetinib (INNO-406) | 2.1 | 2.2 | 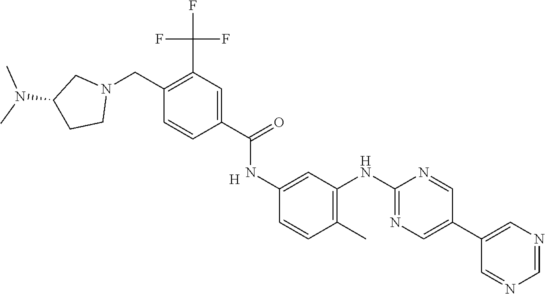 |
| 20 | Imatinib (STI571) | 2.5 | 7.9 | 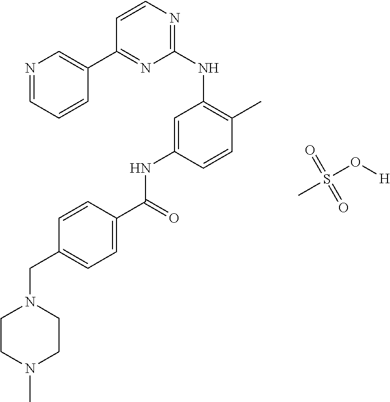 |
| 21 | Mebeverine Hydrochloride | 5 | 10 | 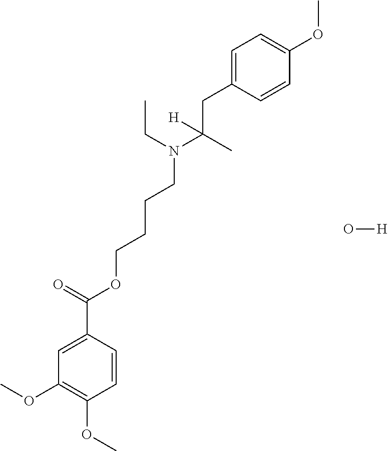 |
| 22 | Trimipramine maleate | 3.8 | 1.5 | 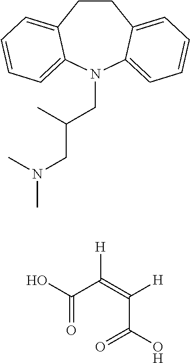 [SMILES: CC(CN(C)C)CN1c2ccccc2CCc2ccccc21 ; O=C(O)/C=C\C(=O)O] |
| 23 | Pentoxyverine Citrate | 5.5 | 3.6 | 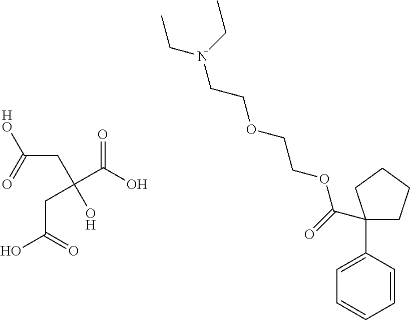 [SMILES: O=C(O)CC(O)(CC(=O)O)C(=O)O ; CCN(CC)CCOCCOC(=O)C1(c2ccccc2)CCCC1] |
| 24 | Cepharanthine | 0.77 | 0.13 | 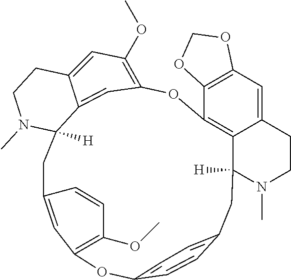 [SMILES: COc1ccc2cc1Oc1ccc(cc1)C[C@H]1c3c(cc4c(c3Oc3cc5c(cc3OC)CCN(C)[C@@H]5C2)OCO4)CCN1C] |
| 25 | Elbasvir | 0.17 | >10 | 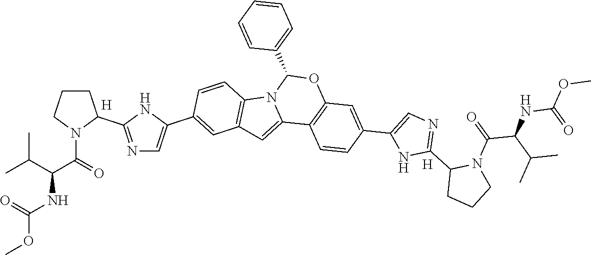 |
| 26 | Erythromycin Cyclocarbonate | >10 | ND | |
| 27 | Remdesivir | 0.96 | 0.1 | |
| 28 | Cediranib (AZD2171) | 0.5 | 3.6 | 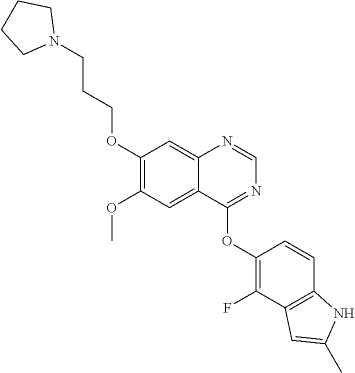 [SMILES: COc1cc2c(Oc3ccc4[nH]c(C)cc4c3F)ncnc2cc1OCCCN1CCCC1] |
| 29 | Lapatinib | 3.1 | 1.6 | 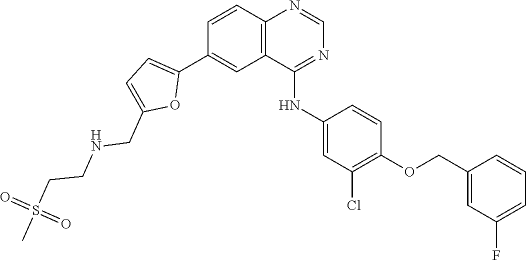 [SMILES: CS(=O)(=O)CCNCc1ccc(-c2ccc3ncnc(Nc4ccc(OCc5cccc(F)c5)c(Cl)c4)c3c2)o1] |

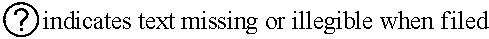

Many of the identified drugs are anti-psychotic and anti-allergic drugs that share a similar tricyclic structure, in agreement with the previous identification of olanzapine (a tricyclic anti-depressant) as an anti-SARS-CoV-2 drug.

The effect of several drugs on the production of viable progeny viruses was evaluated. Cells were treated with 10 μM of the drugs for 2 hours, infected at an MOI of 0.5 and the supernatant collected 2 days after for titration (FIG. 2C). Cepharanthine and azelastine completely eliminated SARS-CoV-2 progeny production (>5-logs decrease), while flupenthixol and desloratidine reduced viral titers by about 2-logs. Ultimately, the screen identified 20 safe-in-human drugs that are able to inhibit both OC43 and SARS-CoV-2 infection in vitro.

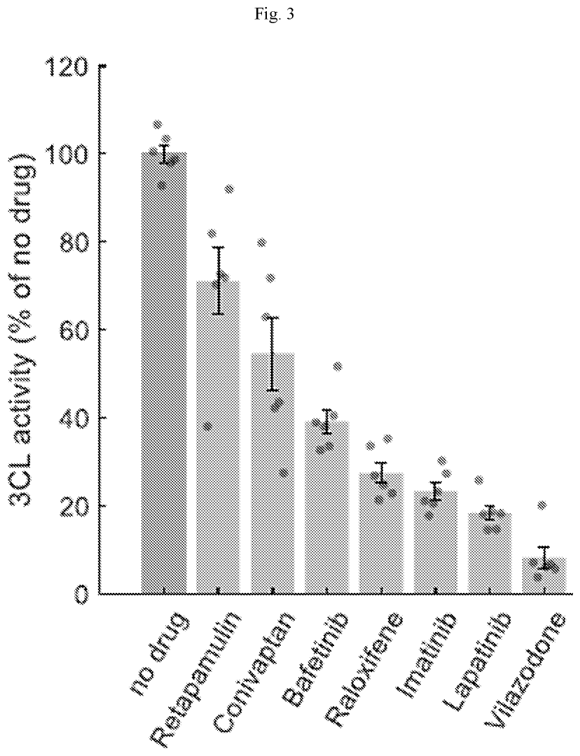

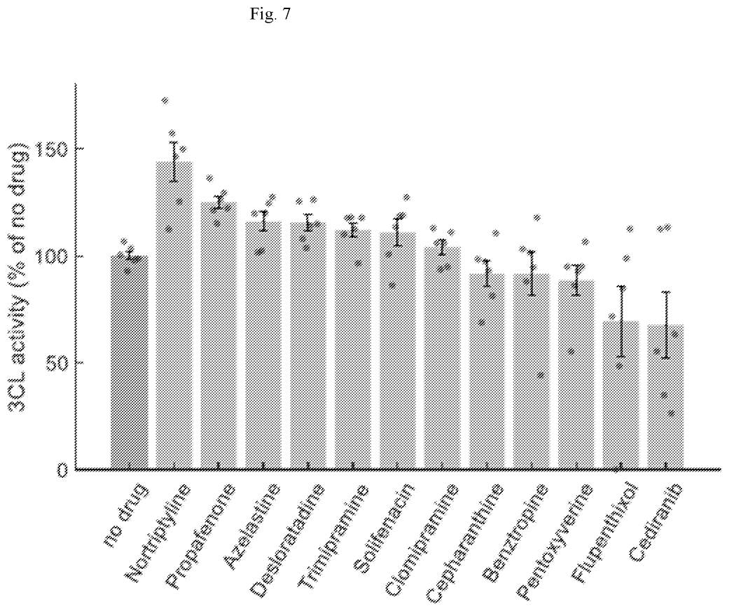

The ability of the drugs to inhibit the SARS-CoV-2 main protease (also known as 3CLpro, Mpro and nsp5) was investigated. 3CLpro is indispensable for the viral replication cycle and is well conserved among coronaviruses. The ability of 20 drugs that inhibited both SARS-CoV-2 and OC43 infection were tested with regard to inhibition of 3CLpro activity in 293T cells transfected with a FlipGFP reporter system (Anand et al., Science, 300: 1763-1767 (2003), incorporated by reference herein) at a single concentration of 10 μM. In this assay, 3CLpro cleavage of the FlipGFP reporter is needed to produce GFP fluorescence, and thus the level of GFP+ cells reports on 3CLpro activity. Eight drugs showed a statistically significant decrease in the percentage of GFP-expressing cells: retapamulin, conivaptan, bafetinib, raloxifene, imatinib, lapatinib, and vilazodone (FIG. 3, FIG. 7).

In addition to azelastine (a nasally administered antihistamine used to treat rhinitis), which inhibits both OC43 (FIGS. 1A-1C) and SARS-CoV-2 (FIGS. 2A-2C), several of the identified hits in the primary screen against OC43 are also administered topically to either the nose or lungs (Tables 1A and 1B). These include salmeterol, budesonide, beclomethasone, fluticasone, and xylometazoline.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing a nidovirus or picornavirus infection in a subject, the method comprising administering to a subject in need thereof an effective amount of an active agent of butamben, butylparaben, conivaptan, amphotericin B, pentoxyverine, lapatinib, vilazodone, imatinib (STI571), benztropine, raloxifene, solifenacin, retapamulin, bafetinib (INNO-406), imipramine, trimipramine, tolterodine, clomipramine, velpatasvir, cediranib (AZD2171), azelastine, desloratadine, nortriptyline, propafenone, mebeverine, elbasvir, cepharanthine, flupenthixol, or a pharmaceutically acceptable salt thereof.

2. The method of claim 1, wherein conivaptan is conivaptan HCl, pentoxyverine is pentoxyverine citrate, vilazodone is vilazodone HCl, benztropine is benztropine mesylate, raloxifene is raloxifene HCl, solifenacin is solifenacin succinate, imipramine is imipramine HCl, trimipramine is trimipramine maleate, tolterodine is tolterodine tartrate, clomipramine is clomipramine HCl, azelastine is azelastine HCl, nortriptyline is nortriptyline HCl, mebeverine is mebeverine HCl, and flupenthixol is flupenthixol dihydrochloride.

3. The method of claim 1, wherein the active agent is azelastine.

4. The method of claim 1, wherein the virus is a nidovirus and the nidovirus is a coronavirus.

5. The method of claim 4, wherein the coronavirus is a beta-coronavirus.

6. The method of claim 5, wherein the beta-coronavirus is SARS-CoV-2.

7. The method of claim 1, wherein the method is a method of treating.

8. The method of claim 1, wherein the subject is a human.

9. The method of claim 1, wherein the active agent is azelastine, and the azelastine is azelastine HCl.

10. The method of claim 9, wherein the virus is a nidovirus and the nidovirus is a coronavirus.

11. The method of claim 10, wherein the coronavirus is a beta-coronavirus.

12. The method of claim 11, wherein the beta-coronavirus is SARS-CoV-2.

13. The method of claim 9, wherein the method is a method of treating.

14. The method of claim 13, wherein the subject is a human.

15. The method of claim 12, wherein the method is a method of treating.

16. The method of claim 15, wherein the subject is a human.
